(19) United States
(12) Patent Application Publication
(10) Pub. No.: US 2015/0160245 A1
(43) Pub. Date: Jun. 11, 2015

(54) PPM QUANTIFICATION OF IODATE USING PAPER DEVICE

(71) Applicants: Marya Lieberman, South Bend, IN (US); Nicholas Myers, South Bend, IN (US); Christopher Sweet, Granger, IN (US)

(72) Inventors: Marya Lieberman, South Bend, IN (US); Nicholas Myers, South Bend, IN (US); Christopher Sweet, Granger, IN (US)

(21) Appl. No.: 14/533,746

(22) Filed: Nov. 5, 2014

Related U.S. Application Data
(60) Provisional application No. 61/899,910, filed on Nov. 5, 2013.

(51) Int. Cl. G01N 33/84; G06T 5/00; G06K 9/62; G01N 21/78; G06T 7/40

(52) U.S. Cl. CPC G01N 33/84; G01N 21/78; G06T 7/408; G06K 9/6267; G06T 5/00; G01N 2201/04; G01N 2021/752

(57) ABSTRACT

A highly sensitive method that utilizes a Paper Analytical Device (PAD) which measures part per million (ppm) levels of iodate iodometric titration is provided. The PAD quantifies concentrations of 0.6-15 parts per million (ppm) of iodine. The PAD has at least 12 reaction zones that contain dried reagents thereon and at least one electronically readable information zone. Salt and water are mixed and drops are placed onto 12 reaction zones that contain the loaded dry reagents. The PAD is shaken and the reaction zones turn blue if a preset iodate concentration has been exceeded. Test results are analyzed by comparing the PAD or an image of the PAD with standards, either by eye or with image analysis software to measure the color intensities which are compared to a calibration curve to quantify the iodate levels. The image analysis quantifies iodine with an average absolute accuracy and precision of 0.9 ppm over a range of 0.6-15 ppm.

### CROSS-REFERENCE TO RELATED APPLICATIONS

This application claims benefit of and priority to U.S. Provisional Application No. 61/889,910, filed Nov. 5, 2013, the entirety of which is incorporated herein by reference.

### BACKGROUND OF THE INVENTION

1. Field of the Invention

The present invention relates to a device for detection and quantification of parts per million (ppm) iodate levels in fortified salt via iodometric titration on a Paper Analytical Device (PAD).

2. Description of the Prior Art

User-friendly analytical devices such as Paper Analytical Devices (PADs) are known in the art as convenient and inexpensive means for assaying chemicals. As these devices contain all necessary reagents and do not require power, they are easy to operate in a field setting.

U.S. Provisional Pat. No. 61/899,910: “Paper Analytical Device for Quantifying Iodine” is the first legal disclosure describing the invention and the information herein.

U.S. Pat. No. 6,136,549 discloses systems for conducting spectrophotometric analysis which includes a chromatographic medium such as an assay test strip that is designed to be contacted with a test solution having activated magnetic particles.

U.S. Pat. No. 6,770,487 discloses “dip stick” style paper-based diagnostic test devices, in which identifying information and the test result are machine-readable.

U.S. Pat. No. 6,847,451 discloses apparatuses for determining the concentration of an analyte in a physiological sample, which include at least one light source, a detector array, means for determining whether a sufficient amount of sample is present on each of the plurality of different areas, and means for determining the concentration of the analyte based on the reflected light detected from those areas determined to have sufficient sample.

U.S. Pat. No. 7,344,081 discloses a method of automatically detecting a test result of a probe zone of a test strip comprising capturing an image of a one-dimensional bar code and an image of at least one test strip from a scanning object, and determining a setting value for the at least one test strip based, at least in part, on said captured image of said bar code.

U.S. Pat. No. 7,885,444 discloses a method for determining a response of each probe zone on a test strip by selecting an average pixel value of each section of reference white respectively adjacent to the image of a target line to serve as a reference for determining a color response of the target line.

US Patent Publication No. 2008/0012083 discloses an analytical system-on-a-chip that can be used as an analytical imaging device for example for detecting the presence of a chemical compound, which can also include software that can detect and analyze the output signals of the device.

US Patent Publication No. 2011/0189786 discloses a method of detecting the presence or absence of an analyte in a fluid sample. The method includes applying the sample to an inlet zone of a diagnostic system that includes a hydrophilic cotton loading thread to serve as a capillary to deliver a solute to a reagent testing zone and detecting color change of reagent analyte interaction.

US Patent Publication No. 2011/0111517 discloses a paper-based microfluidic assay device comprising a porous, hydrophilic substrate; a fluid-impermeable barrier defining a boundary of an assay region and a boundary of a main channel region, the main channel region fluidically connected to the assay region; and a strip of conductive material disposed on the porous, hydrophilic substrate for detecting the concentration/flow of analyte.

In a commercial embodiment, pSiFlow Technology Inc. provides a mobile testing and process management web infrastructure built around its Calibrated Color Match (CCM) image processing technology that enables digital reading of color-based test strips using any mobile phone with a camera.

US Patent Publications Nos. 2013/0034908 and 2014/0051173 each disclose an analytical device for detection of at least two chemical components indicative of a low quality pharmaceutical product. The analytical device includes at least two assay regions, at least one assay reagent or precursor thereof in the assay regions, and at least one electronically readable information zone.

Some medical conditions arise not from a pathogen but from a deficiency in an essential nutrient. For example widespread iodine deficiency is a problem in many underdeveloped countries that is associated with developmental impairment in children. Fortification of table salt with potassium iodate or potassium iodide is a common, but not the only route, to address this problem. However, production and distribution methods for iodized table salt in many developing countries yield inadequate or inconsistent levels of iodine. Unfortunately the time and expense of testing for iodized table salt deters manufacturers, distributors, and end-users from testing iodized table salt to determine iodine concentrations. Thus a low-cost method of testing iodized table salt at the production facility or in the field is needed to determine whether the salt is adequately fortified with iodine within therapeutic concentrations recommended by the WHO. At production, salt is typically iodized at levels around 30-50 parts per million of iodine per kg of salt (ppm) with potassium iodate or iodide. The main iodizing agent used in developing countries is potassium iodate, which is less susceptible to degradation in moist salt than potassium iodide. Regardless of which iodizing agent is used, the iodine content of the salt degrades over time in storage. To be therapeutically effective, table salt must contain at least 15 ppm of iodine at the time of consumption so salt in all steps of the supply chain from production to tabletop must be carefully monitored for proper iodization levels.

Current methods for iodine measurement in fortified salt include glassware titration, rapid test kits, and spectroscopic test kits. Each method requires trade-offs in terms of accuracy, cost, and ease of use. Titration has been used for quantification of iodine for over a century, and when conducted by a skilled operator in a well-controlled lab setting, titration gives excellent accuracy and precision. However under field conditions in developing world settings such as in small salt fortification factories, the accuracy and precision are compromised by practices such as use of old or impure reagents, lack of calibration or service for analytical balances, and use of over-concentrated titrant. Rapid test kits in which test reagents are applied directly from dropper bottles to solid salt to detect the presence of iodate are inexpensive. Notwithstanding this advantage, an external validation study showed that the tests do not give reliable quantification. In the past decade, spectrophotometric assays using the characteristic purple color of the starch-tri-iodide complex have been utilized to monitor salt quality but these assays require purchase of both a specialized reader and kits of the necessary reagent chemicals.

Thus there still exist long-felt needs for a low-cost, easy-to-use, reliable, minimalistic and highly sensitive chemical means of detecting iodine in dietary supplements such as iodized salt. The present invention addresses these needs by providing an inexpensive, user-friendly, consistent, highly sensitive analytical device capable of detecting and measuring parts per million (ppm) levels of iodine in fortified iodized salt.

### SUMMARY

The present disclosure provides an easy-to-use, inexpensive, highly sensitive analytical device, and methods of use thereof, with superior chemical parameters for detection and analysis of part per million quantities of an analyte, typically iodate used as a nutritional supplement. The analytical device typically comprises a porous hydrophilic medium; a plurality of reaction zones associated with the porous hydrophilic medium; at least one assay reagent deposited and subsequently dried in each of the reaction zones; and at least one electronically readable information zone which provides color information necessary for identification of the device, analysis, detection and quantification of the nutritional supplement. Also, each reaction zone can be further divided into a plurality of separate loading zones, wherein each loading zone has a different dried assay reagent applied thereon. This feature allows deposition and storage of reagents that are incompatible. Typically, the porous hydrophilic medium is paper, such as Ahlstrom 319 blotting paper. Preferably the at least one electronically readable information zone comprises a plurality of fiducial markers for transforming and correcting a captured image of the analytical device, as well as color standards to facilitate analysis and processing of the color information to more accurately detect and quantitate the nutritional supplement.

In one embodiment, the alignment references include a plurality of fiducial markers for orienting the captured image, and the analytical device further includes at least one of an identification tag such as a two-dimensional barcode and a color calibration zone.

In a preferred embodiment, the analytical device further includes a color calibration zone and the method further comprises automating the color analysis by capturing an image of the analytical device using a camera; and providing image analysis software capable of recognizing and quantifying a color change within the reaction zones of the analytical device that is shown in the captured image. The analytical device typically comprises a plurality of fiducial markers for orienting the captured image such that the image software can correct and transform the captured image based on these fiducial markers, thereby aligning and scaling the captured image, read test results from the reaction zones in the transformed image, and analyze and classify the test results. Preferably the method further comprises providing the image analysis software on the camera-device for processing the captured image in situ, or on a network server to process the captured image by sending the image to the network server that performs the analysis and transmitting detection results back to the camera-device.

The present disclosure also provides an analytical device, which is typically a PAD for analyzing and quantitating parts per million concentrations of a component of a food product such as but not limited to fortified iodized table salt. The analytical device typically comprises a porous hydrophilic medium, typically paper; a plurality of reaction zones associated with the porous hydrophilic medium; and at least one dried assay reagent in each of the reaction zones, wherein the at least one assay reagent is capable of identifying a component of the food product, and is present at different concentrations in each of the reaction zones so as to facilitate a highly sensitive quantitative analysis of the component. The analytical device can include a hydrophobic barrier that is present to define a reaction zone. The reaction zone can be further divided into many sections by multiple hydrophobic barriers of varying sizes.

The analytical device can also include at least one electronically readable information zone which after activation of the device provides color information necessary for quantitative analysis of the component; and a color calibration zone. Preferably the at least one electronically readable information zone comprises affixed fiducial markers thereon for transforming and correcting a captured image of the analytical device to facilitate analysis and processing of the color information in order to more accurately detect the quantity of the food product.

In the preferred embodiment, the food product is preferably an iodized salt, and the iodine components to be analyzed are selected at least one of iodate (IO₃⁻) and iodide (I⁻).

The analytical device can be used by submerging all or a portion of the device in a solution containing or suspected of containing a food product to be analyzed. The analytical device can be used by applying a solution containing, or suspected of containing, a food product to be analyzed. If a food product is applied to the analytical device, the food product can be applied to all or only a portion of the analytical device. If the food product is applied to the analytical device, it is usually applied as a solution. When a solution is applied to the reaction zone, the solution can flow over the thin barriers on the analytical device but can remain contained by the outside boundary, thereby forming a solution dome. The solution dome allows the reagents dried onto the analytical device to dissolve and provides a pathway for the reagents to meet and initiate the chemical reactions.

However in other embodiments, the methods and compositions described herein can also be used to perform other iodometric titrations such as but not limited to the back-titration used in the quantification of beta-lactam antibiotics for detection of substandard pharmaceuticals.

### BRIEF DESCRIPTION OF THE DRAWINGS

FIG. 1 depicts a preferred embodiment comprising twelve assay reaction zones and locations for chemicals placed onto the PAD.

FIGS. 2A)-2L) depict the PAD response to various levels of iodine from iodate.

FIG. 3 demonstrates calibration curves of the PAD response to different levels of iodine. Each calibration curve represents a reaction zone that contains different amount of thiosulfate.

FIG. 4 depicts the accuracy of the PAD method.

FIG. 5 illustrates a Bland-Altman plot comparing the PAD to the known iodine concentration.

FIG. 6 depicts the stability of the PAD device.

### DETAILED DESCRIPTION

The claimed invention is not limited in its application to the details of construction and to the arrangements of the components set forth in the following description or illustrated in the appended Figures. The claimed invention is capable of other embodiments and of being practiced and carried out in different ways. Also it is to be understood that the terminology used herein is for the purpose of description and should not be regarded as a limiting factor.

As such, those skilled in the art will appreciate that the conception upon which this disclosure is based can also be utilized as a basis for designing other structures for carrying out the several purposes of the claimed invention. It is, therefore, equally important that the claims be regarded as including such equivalent constructions insofar as they do not depart from the spirit and scope of the present disclosure.

As used herein, the term “Paper Analytical Device” or “PAD” refers to a composition based on a porous hydrophilic material (such as paper) and comprising areas of hydrophobic barriers which define hydrophilic assay reaction zones.

As used herein, the term “camera device” refers to a device that contains a camera component. Exemplary camera devices are various types of digital cameras and scanners as well as mobile devices such as cell phones, smartphones or similar devices. When digital cameras are used, the images can be uploaded to a computer or other electronic device that is capable of transmitting the pictures to another location. When a picture or image is taken by a camera device integrated into a mobile phone, the phone already generally has the ability to forward the image, e.g., as a text message or as part of an e-mail.

As used herein, the term “fiducial marker” refers to an object that is placed in the field of view of an imaging system, which appears in the image produced for use as a point of reference or a measure.

As used herein, the term “gray scale” refers to an image in which the digital value of each pixel is a single sample number, that is, it carries only intensity information.

As used herein, the term “substandard pharmaceutical” refers to products purported to be genuine products for treating a disease or disorder but which contain low concentrations of active ingredients that are not sufficient to treat purported use or that fail to meet regulatory standards, a product containing substitute active ingredients that can have undesired side effects compared to the stated drug, a product containing active ingredients that have undergone degradation, or a product having no active ingredients at all. In addition, the term includes products that contain ingredients that should not be present in a genuine product of the stated type including ingredients that can be toxic.

As used herein, the term “Equivalence Point” refers to the point at which the number of moles of an added titrant is stoichiometrically equal to the number of moles of the analyte present in the sample. Thus it is the smallest amount of titrant that is sufficient to fully neutralize or react with the analyte.

As used herein, the term “limit of detection” refers to the lowest quantity of a substance that can be detected and distinguished but not quantified from the absence of that substance.

As used herein, the term “Lower limit of quantification” refers to the lowest quantity of a substance that can be quantified and distinguished from the absence of that substance.

As used herein, the term “accuracy” refers to the degree of closeness of measurements of a quantity to that quantity's actual (true) value.

As used herein, the term “precision” refers to reproducibility and repeatability, which is the degree to which repeated measurements under unchanged conditions show the same results.

As used herein, the term “robustness” refers to the persistence of a systems' characteristic behavior under perturbations or unusual conditions.

As used herein, the term, “Coefficient of Determination,” denoted R² refers to how well data fit a statistical model. It provides a measure of how well observed outcomes are replicated by the model. A coefficient of determination R² value can range between 0 and 1, wherein 1 is a perfect fit for a statistical model, whereas 0 represents a non-fit between two variables.

As used herein, the term “standard deviation” refers to the amount of variation or dispersion from the calculated average.

As used herein, the term “spiking” refers to iodate levels with known concentrations are added directly to the aliquots of an analyzed sample in order to determine the accuracy and precision of the PAD method.

As used herein, the term “PAD” should be understood to be used interchangeably with the term “saltPAD”.

As used herein, the term “affine transformation” refers to a function between affine spaces which preserve points, straight lines and planes. Also sets of parallel lines remain parallel after an affine transformation. An affine transformation does not necessarily preserve angles between lines or distances between points though it does preserve ratios of distances between points lying on a straight line.

As used herein, the term “perspective transformation” refers to the conversion of a 3 dimensional image into a 2 dimensional image.

As used herein, the term “canonical coordinate system” refers to sets of coordinates which can be used to describe a physical system at any given point in time.

As used herein, the term “beta-lactam” covers any four-membered lactam antibiotics such as but not limited to ampicillins, penicillins, cephalosporins, carbapenems and monobactams.

As used herein, the term “iodometric titration” generally refers to the iodometric titration of iodate or the detection of iodide as depicted in (i)-(iv) hereinbelow. In the current description, the thiosulfate levels required to detect specific quantities of iodate are not stoichiometric as would be expected to one of ordinary skill in the art. The titrations are characterized by the following reactions:

IO₃⁻+6H⁺+8I⁻ [symbol] 3I₃⁻+3H₂O (i)

4H⁺+3I⁻+2NO₂⁻ [symbol] I₃⁻+2H₂O+2NO (ii)

I₃⁻+2S₂O₃²⁻ [symbol] 3I⁻+S₄O₆²⁻ (iii)

I₃⁻+starch [symbol] Blue complex (iv)

The present disclosure provides an easy-to-use, inexpensive analytical device such as a PAD and methods of use thereof for detection of ppm levels of iodate or iodine in fortified salt. In accordance with preferred embodiments, a highly sensitive analytical device, which is preferably a PAD, and methods for detecting and quantifying iodate levels in fortified salt are disclosed, wherein the method shows superior analytical parameters such as but not limited to limit of detection (LOD), lower limit of quantification (LLOQ), accuracy, precision, robustness and stability. The results are either quantified by visual inspection, or in a more sophisticated manner and more accurately with a camera-enabled device and image analysis program. Thus after using a PAD the user can take a picture and can have a smart phone application analyze the results. Alternatively the picture can be transmitted to a server and be analyzed by a software program that interprets the PAD results. In alternative embodiments the quantification of low-quality substandard pharmaceuticals such as but not limited to beta-lactams can similarly be quantitated by the described PAD method.

### Porous Hydrophilic Substrate

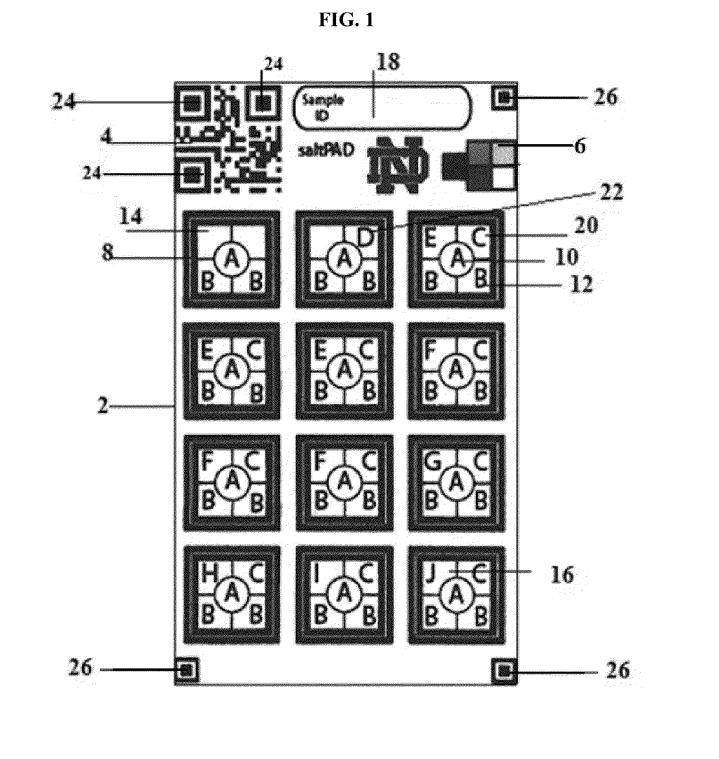

As shown in FIG. 1, typically the analytical PAD (2) comprises a porous hydrophilic medium, which is preferably a paper such as a fast chromatography paper or an absorbant blotting paper. A suitable porous hydrophilic medium is one that has enough absorption capacity to hold adequate amounts of reagent. Moreover, it has durability and stability such that it does not fall apart or fall over when it is wet and is compatible with at least one of the methods used to fashion the reaction zones (8). Characteristics which should be considered when designing an analytical device such as the PAD (2) include printability, density, thickness, pH, basis weight, wetability, compatibility with fabrication methods, pore size and porosity. Preferred characteristics include a substrate that is printable, flexible but sturdy enough to resist tearing, a medium thickness around about 0.4-0.6 mm, pH relatively close to neutral, a medium density and weight, resistance to deterioration caused by fabrication methods and materials and a large pore size. Examples of suitable materials for an analytical device such as a PAD (2) include but are not limited to nitrocellulose acetate, chromatography paper, cellulose acetate, cellulosic paper, filter paper, tissue paper, writing paper, paper towel, cloth, and porous polymer film. In one embodiment, the substrate for a PAD (2) is Whatman 3 mM Chr chromatography paper, Ahlstrom 205, Ahlstrom 222, Ahlstrom 226, Ahlstrom 319, or Whatman No. 1 filter paper. In one preferred embodiment, Ahlstrom 319 filter paper is used.

The basis weight (g/m²) of the PAD (2) can range from about 50 g/m² to about 400 g/m²; including 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400 g/m². Specific preferable basis weights include about 90, 176, 187, 192, and 307 g/m². Thickness of the PAD (2) can range from about 1.0 mm to about 0.10 mm thick, including 0.1, 0.2, 0.3, 0.4, 0.5, 0.6, 0.7, 0.8, 0.9, and 1.0 mm. Specific preferable thickness include 0.18, 0.47, 0.48, 0.69, and 0.83 mm. The PAD (2) can have a pH ranging from about 6.0 to about 8.0, including having a pH of 6.0, 6.5, 7.0, 7.5, and 8.0. Specific preferable pH values can include 5.81, 5.99, 6.25, 6.31, and 7.09. Pore size can range from about 10.0 micrometers to about 30.0 micrometers, including 10, 11, 12, 13, 14, 15, 16, 17, 18, 19, 20, 21, 22, 23, 24, 25, 26, 27, 28, 29, and 30 micrometers. Specific preferable pore sizes include about 11.0, 19.0, and 22.0 micrometers. The most preferable basis weight is at least about 192.0 g/m², the most preferable thickness is at least about 0.48 mm, the most preferable pH is at least about 5.99 and the most preferable pore size is at least about 19.0 micrometers.

### Fabrication of the PAD and Reaction Zones

The final dimension of the PAD (2) can have a length “L”, width “W” and height or depth “H” that can vary all depending on the number and size of reaction zones (8) and information zones needed on the PAD (2). For example in one embodiment, the PAD (2) can be 1.75 inches long, by 3 inches wide. In another embodiment, the PAD (2) can be 5 inches long by 3 inches wide. The height or depth is determined by the specific paper substrate and fabrication method used. In one embodiment, an inert backing material is required, which will increase the PAD (2) depth. In another embodiment, laminated or layered structures are produced which again increase the PAD (2) depth. In some embodiments, the PAD (2) can include a region or flap that is designed to be folded over by the user during or after use.

A plurality of assay reaction zones (8) are defined on the analytical device, either by affixing individual strips or other shapes cut from paper onto an inert backing material, or by patterning a piece of paper with a hydrophobic barrier permeating the thickness of the paper medium to define the boundaries of the desired assay reaction zones (8). The hydrophilic assay reaction zones (8) or reaction areas with the boundaries defined by the hydrophobic area can be in any suitable size or shape. Suitable shapes for the assay reaction zones (8) can include rectangular, circles or “spots”, squares, triangles etc. The area of each reaction zone (8) must be sufficient enough to contain the necessary amounts of embedded dried reagents to interact with the chemicals to be detected, and the lane dimensions must be large enough such that the color produced in the chemical reactions can be clearly distinguished by the camera device.

The assay reaction zones (8) of the PAD (2) can be produced in a number of ways that are known to one skilled in the art. For example photolithography of a resist such as SU-8 can be used to produce hydrophobic regions within the hydrophilic paper medium according to the procedures laid out in US patent Application Publication number 2011/0111517 A1 which is herein incorporated in its entirety. In the SU-8 method, the paper medium is saturated with a cross-linking photoresist and then exposed to UV radiation in a pattern creating hydrophobic and hydrophilic zones thereby. Alternatively, the PAD can be fabricated by a heated metal stamp that impregnates selected regions of the paper with parafin wax, according to the procedures laid out in H. Yagoda, Industrial and Engineering Chemistry, 1937, 9(2), 79-82, which is herein incorporated in its entirety. Alternately, the PAD (2) can be fabricated using wax printing according to the procedures laid out in Lu, Y.; Shi, W.; Jiang, L.; Qin, J.; Lin, B. Electrophoresis 2009, 30, p. 1497-1500 and Carrilho, E.; Martinez, A. W.; Whitesides, G. M. Anal. Chem. 2009, 81, p. 7091-7095, which are herein incorporated in their entireties. In a preferred embodiment, an HP Color Qube printer is used to deposit wax ink, preferably black, in the desired regions of Ahlstrom 319 paper according to a template laid out in a computer program such as Adobe Illustrator. Alternatively the template can be stored and printed as an image file such as a PDF.

The preferred paper type is too thick for wax deposition on one side of the paper to form the necessary continuous hydrophobic barrier so wax ink must be printed on both sides of the paper after which the paper is heated to 70-120° C., preferably 100° C., so as to allow the wax to melt through the paper and form a continuous hydrophobic barrier surrounding the desired assay reaction zone (8).

The “cut and paste” method requires the lanes to be cut from the hydrophilic paper medium and adhered to a relatively strong backing, mylar plastic for example, using an adhesive. This method does not require the application of a hydrophobic agent in order to define the hydrophilic reaction areas. As a result the chance of bleed-over of hydrophobic agent into the lanes is eliminated. The paper shapes can be cut using any precise cutter, such as an x-acto-knife, craft cutter, laser cutter, or die cutter.

### Reagents and Reagent Deposition

Now generally referring to FIG. 1, the method of reagent deposition onto the saltPAD (2) will be described in more detail. As shown in FIG. 1, the saltPAD (2) preferably has a sample ID identifier space (18). As explained previously, the saltPAD (2) preferably has twelve assay reaction zones (8), wherein each assay reaction zone (8) is further split into a number of separate loading zones that each have a different dried reagent thereon with a different concentration. In the drawing, 5 separate loading zones are disclosed on each reaction area, but the number can range from 2 to 16 and preferably from 3 to 9 regions, and can include 2, 3, 4, 5, 6, 7, 8, 9, 10, 11, 12, 13, 14, 15, or 16 regions. Various reagents can be optionally loaded into the reaction zones individually either by hand, or via an automated process. The reagents can be loaded as liquid solutions or suspensions, and can be subsequently allowed to dry prior to the use of the saltPAD (2). Table 1 summarizes and demonstrates the identity of the reagents, their concentrations and the location that they are spotted onto the saltPAD (2). The reactions that are being carried out in the reaction zones are standard iodometric titration reactions and control reactions that allow the user to assess whether the PAD is functioning properly. For example as best depicted in FIG. 1, each row on the saltPAD (2) has three separated rectangular reaction zones (8). If the positive control turns blue and negative control remains white, then this demonstrates that the saltPAD (2) is working properly. The remaining reaction zones are used for quantification of the analyte or limit tests. Changing the concentration of the thiosulfate (3.0-150 mM) in locations E, F, G, H, and I will vary the response of the saltPAD (2). Acidification of the reaction mixture is necessary for the titration, which is why all the reaction zones contain p-toluenesulfonic acid (12). However, this reagent can conveniently be exchanged, as any redox inactive acid that is stable during dry storage can be used for the iodometric titration. Starch is used as an indicator for the presence or absence of iodine; this reagent (10) is typically used at a concentration of 2%, but the concentration could be varied. The starch indicator has the sole purpose of making the test more sensitive and is typically pre-loaded in the central part (10) of the twelve reaction zones (8) depicted as “A” in FIG. 1 and Table 1. The sodium nitrite (22) depicted as “D” in FIG. 1 and Table 1 for iodide detection can conveniently be exchanged for any other oxidizing agent. Iodide is necessary for the iodometric titration reaction and for complexation of the resulting iodine as triiodode ion. It is added in the form of potassium iodide at a concentration of 0.5 M, but any amount in stoichiometric excess could be used. Long term storage of iodide requires that it be strictly isolated from acid on the test cards. A small quantity of divalent cadmium (Cd²⁺) (20) identified as “C” is used to stabilize the iodide. Potassium iodate (16) which is loaded in location J is used as the positive control on the saltPAD (2) at a concentration of 625 mM but the concentration only has to exceed 1 ppm after the test solution is applied. The location of the reagents can be varied within each reaction zone.

But it should be understood that the reagents that can be spotted onto the saltPAD (2) are not restricted to only the shown reagents in Table 1. Examples of other reagent materials suitable for use in the PAD (2) include but are not limited to Folin-Ciocalteu, potassium hexacyanoferrate(II) trihydrate, iodine-potassium iodide reagent, universal indicator, phenolphthalein, ferric chloride, tri-iodide, tri-iodide-starch complex, soluble starch; cationic, anionic, and neutral pH indicators; barium chloride, sodium rhodizonate, potassium hexacyanoferrate(II), NaOH or KOH, tosic acid, potassium carbonate, citric acid, copper sulfate, sodium tetraphenylborate, cobalt thiocyanate, ammonium molybdate, nitroaniline, 1,2-napthaquinone-4-sulfonate, dimethylglyoxime, and paradimethylaminobenzaldehyde. These reagents can be deposited onto the PAD (2) from an aqueous solution or from organic solution. For wax printed PADs (2), acetonitrile is the preferred organic solvent because the wax barriers are minimally affected by the acetonitrile. Many colorimetric reagents plateau at particular concentrations so that adding additional reagents will not enhance color results. Thus the upper limit on the amount of reagents added is more or less determined by the PAD's (2) loading capacity. The volume of the reagent loaded onto the PAD (2) can suitably range from about 2 to about 100 microliters, including 2, 3, 4, 5, 6, 7, 8, 9, 10, 20, 30, 40, 50, 60, 70, 80, 90, 100, 11, 120, 130, 140, 150, 160, 170, 180, 190, and 200 microliters. In the specific example depicted in Table 1, 2 microliters of the reagent has been deposited into each loading zone.

Reagents can be deposited on the surface of the PAD (2) in many ways that will be familiar to those having skill in the art including but not limited to the use of: microcapillary pipettes and droppers, single- or multi-channel automatic pipetting devices, rods that can capture a droplet of solution or depositors that perform this function with multiple rods simultaneously, dipping or spraying equipment, or solution deposition robots. In one preferred embodiment, the reagents are manually deposited using an automatic pipette. In another preferred embodiment, the reagents are deposited using a Biomek FX 96-well-plate replicating robot.

### Compositions Suitable for Analysis

The chemicals to be detected can be in any suitable formulation. Any solid formulation must first be dissolved into an aqueous solution or suspension in order to be used with the saltPAD (2). In the preferred embodiment, the saltPAD (2) is used to analyze foodstuffs that have been supplemented or fortified with micronutrients like iodine. However in certain other embodiments, the PAD can equally well be used for other iodometric titrations such as but not limited to the quantification of beta-lactam antibiotics for the detection of low quality substandard pharmaceuticals.

TABLE 1

| Location | Used Reagent |
|---|---|
| A | 2% Starch |
| B | 1.0M p-toluenesulfonic acid |
| C | 0.5M KI/0.3M CdCl₂ |
| D | 0.42M NaNO₂ |
| E | 3.0 mM Na₂S₂O₃ |
| F | 16.0 mM Na₂S₂O₃ |
| G | 30.0 mM Na₂S₂O₃ |
| H | 43.5 mM Na₂S₂O₃ |
| I | 150 mM Na₂S₂O₃ |
| J | 625 mM KIO₃ |
| Blank | No Reagent |

### Deposition of the Composition to be Analyzed on the PAD

As shown in FIG. 1, in accordance with the preferred embodiment, a saltPAD (2) has been created that can quantify iodate in fortified salt. The salt sample requires a minimal amount of preparation. In accordance with the preferred embodiment, fortified salt is first dissolved and drops of the solution are applied onto the twelve rectangular reaction zones (8) on the saltPAD (2) that contain the loaded dried reagents as depicted in Table 1. Each large rectangular reaction zone (8) is further subdivided by narrow wax lines thereby forming five loading zones. The drops are applied to form a dome of solution that connects all five spaces within each reaction zone square (8). The saltPAD (2) is subsequently shaken for at about three minutes during which time the reaction zones (8) turn blue if a preset iodate concentration has been exceeded. Suitably one part salt and five parts of water are mixed in order to make a 1:5 dilution before applying the solution onto the saltPAD (2). But generally any dilution factor from 1:2 to 1:10 or greater can be chosen as desired just as long as the color development is not compromised, and the color development further falls within the calibration ranges and the specific dilution factor is subsequently multiplied so as to obtain the correct iodate amount. As mentioned above, in the preferred embodiment, up to five reagents can be stored without mixing in the five loading zones. When the solution is applied to the reaction area, the solution forms a “dome” confined by the solution meniscus and pinned at the border of the hydrophobic reaction zone (8). The reagents stored in the five loading zones subsequently dissolve and mix, which is effectuated by a surface-tension enabled mixing mechanism (STEM).

Once the composition has been applied to the reaction zones (8), the disposing of the solution into the reaction zones (8) typically causes a colorimetric change in each reaction zone as previously outlined in reaction equations (i)-(iv) that can be analyzed in order to quantify the amount of the targeted chemical.

The hydrophobic regions can also define control regions within the hydrophilic paper medium. A negative control (14) can be included in order to verify that the PAD (2) has not become contaminated during storage or use or that the sample matrix or solvent used to prepare the samples does not interfere with a color generating reaction. A positive control (16) can be included to show that the reagents in a reaction zone (8) are still viable, or it can be used as a standard for the image analysis software as disclosed hereinbelow. The saltPAD (2) can also contain reaction zones (8) whose function is to demonstrate that the user has complied with instructions for correct use of the saltPADs (2), or reaction zones (8), whose function is to demonstrate that the PAD (2) is an authentic device and is not a counterfeit.

In alternative embodiments when beta lactam antibiotics are to be analyzed for the detection of low quality substandard pharmaceuticals, a solid formulation can first be crushed or ground to powder, or a capsule containing powdered antibiotics can be opened up and subsequently dissolved in water as explained above before being applied drop-wise onto the desired reaction zones on the saltPAD (2). In some embodiments, samples are prepared using conventional glassware according to the methods described in USP Method <425>, which is herein incorporated in its entirety, but instead of utilization of a glassware titration, the samples are applied directly to the reaction zones of the PAD.

### Information Identification Zone

The saltPAD (2) contains at least one electronically readable information zone which provides information necessary for determining the outcome of the test performed on the saltPAD (2) based on images obtained by a camera device. The information zone typically includes appropriate information that is electrically readable per se or after being photographed, or otherwise imaged electronically. As demonstrated in FIG. 1, such information can include an identification tag such as a two-dimensional bar code (e.g., a QR code) (4), a color calibration zone (6) and a plurality of fiducial (24) markers.

As shown in FIG. 1, each saltPAD (2) can be imprinted with a two-dimensional barcode (4) such as a Quick Response (QR) barcode (4) that contains the type and serial number of the PAD (2) such that a PAD (2) test can be uniquely identified and the necessary color processing steps to perform the test can be automatically determined, which provides a simple and inexpensive way to uniquely identify the saltPAD (2) in addition to providing pertinent information for perspective distortion correction and subsequent color analysis. Depending on the application, other information can also be encoded in the two-dimensional bar code image (4). A key task of the image analysis software is the perspective correction and transformation of distorted images, which transforms an image captured at an unknown standoff and optical axis position to a canonical coordinate system in which regions to be analyzed for subsequent color characterization are expressed. The origin and basis vectors for this coordinate system can be automatically calculated from the position of “finder marks”, e.g. plurality of fiducial markers (24) on the QR bar code (4). As shown in FIG. 1, in some embodiments, each saltPAD (2) can also contain one or more additional fiducial markers (26) in the remaining three corners of the saltPAD (2), opposite the corner where the two-dimensional bar code is located. The identification zone can in theory be placed anywhere on the saltPAD (2). Preferably the identification zone can be printed on the saltPAD (2) prior to application of hydrophobic regions and the identification zone is located on an upper corner of the saltPAD (2).

### Color Calibration Zones

Although test results can be read by eye by comparing the PAD or an image of the PAD to images of PADs that have been run with standard concentrations of an analyte, they can also be read using an image processing program. To get a more precise quantitation of target chemical determination in analyzing a PAD (2), the color content of specific regions of the PAD (2) can preferably be analyzed to automatically determine the test result. This removes human subjectivity and inherent bias in color interpretation. However PAD (2) images can be captured under different ambient lighting conditions or with different camera-devices, and the effect of these perturbations on PAD (2) color distributions have to be suppressed. Thus it is important to perform color calibration using the color calibration zone (6) depicted in FIG. 1 on the saltPAD (2), which consists of different colored sub-regions, including a white region and a black region. Image analysis software can be used to compare the extracted colors in the saltPAD (2) image's color calibration zone to known values in order to identify the specific color correction methods needed. One such method is white balancing, in which the overall brightness of the image is adjusted to force the white square in the PAD (2) image to have a pure white color value. The calibration zone (6) can practically be in any suitable shape including but not limited to rectangles, squares, circles, or triangles. The sub-regions can be in any suitable shape, including rectangles, squares, circles or triangles. Preferably the color calibration zone (6) is a rectangle region and the sub-regions are different colored squares. The color calibration zone (6) can be printed onto the PAD (2) prior to or after application of hydrophobic regions. The calibration zone (6) can suitably be placed anywhere on the PAD (2). Preferably the calibration zone is printed on the PAD (2) prior to application of hydrophobic regions and is located on an upper corner of the PAD (2). The color intensities of the calibration zones would typically be used to correct the color intensities read from reaction zones before comparison to a stored calibration curve.

In the preferred embodiment, the PAD (2) comprises one information zone having a color calibration zone (6), another information zone having a plurality of fiducial markers (24), and yet another information zone comprising a QR code (4) or other identification tag.

The method further comprises providing a camera device capturing an image of the PAD (2) that has reacted with the composition using the camera device, and providing an image analysis software capable of using information provided by the information zone and the image of the test result in order to identify and quantify a colorimetric change within the test reaction zone (8) of the PAD (2) shown in the captured image. In the preferred embodiment, the captured image contains a two-dimensional bar code such as a QR code (4) and a plurality of fiducial markers (24). The image software identifies the QR code region (4), and separates the image of the PAD's (2) reaction zones (8) from the background present in the picture, scales, rotates, and performs geometrical transformations on the captured PAD (2) image based on the QR code (4) and the plurality of fiducial markers (24), and subsequently classifies the test results. The method further comprises compiling a database of the captured images of the PAD (2) and the computed test outcomes, wherein the two-dimensional barcode is a QR code (4) that allows for automated identification of a specific PAD (2), serial number and fabrication date.

In one embodiment, the image analysis software is provided on the camera device for processing the captured image in situ. Alternatively the image analysis software can be provided on a network server such that the captured image is processed by sending the picture to the network server that performs the analysis and transmits the results back to the camera-device.

### Achieving Quantification by Computer Analysis

Computer analysis has the potential to allow for a more refined quantification method than by analysis by eye. A program has been set up that locates the blue indicator circles (10) and measures the gray scale intensity. These intensities are compared to calibration curves in order to calculate the iodine concentration in the sample.

The necessary processing can be implemented on the same phone used to capture the image, or on a server system accessible on the Internet, which preferably receives the image via a text message or SMS from the sending phone. Each PAD (2) has a plurality of fiducial marks (24) printed on it, so as to allow image analysis software to automatically orient the card image for processing. At a minimum, a PAD (2) will contain a QR code block (4) with at least three embedded “finder markers” characterized by being fiducial markers (24). It can contain more than one QR code block (4), isolated fiducial markers (26) not included in the QR code block (4), or a combination of QR code blocks (4) and isolated fiducial markers (24). The software extracts the position of each fiducial marker (24) and develops one or more normalization transformations that produces a new image, in which the PAD (2) cards' elements including the fiducial markers (24) and the indicator circles (10) are in fixed and known positions. This normalization step could conveniently employ a single affine transformation, a single perspective transformation or a combination of one or more affine and/or perspective transformations.

The image color information within each indicator circle (10) is extracted from the normalized image, and then compared against one or more reference or standard colors in order to determine the iodine concentration level. This step of processing can include the use of a location refinement technique to precisely localize the indicator circle position (10). The location refinement technique can include image template matching and localization using a predefined image of the indicator circle and its surroundings as a template. The color comparison can be a comparison in RGB (red, green, blue) intensity coordinates, CIELAB color coordinates or another color coordinate system. The reference colors to which the indicator circle (10) color is compared can be functions of the color measurement at one or more positions on the normalized PAD (2) image. The functions can include black-level correction, white-balancing, both or another color correction algorithm. Alternatively the reference colors can be pre-specified colors embodied in the software and not extracted from the image.

Regardless of the technique, the proximity between the indicator circle color (10) and the reference colors is the basis for estimating the iodine amount.

### Controls

The hydrophobic regions can also define control regions within the hydrophilic paper medium. For example a timer region can be included in order to alert the user when the test has completed. Additionally the PAD (2) can include positive (16) or negative control regions (14). A negative control (14) can be included in order to verify the purity of the reaction solvent. A positive control (16) can be included in order to verify the presence of the chemical to be detected. The control substrates are included as dried reagents on the PAD (2). The PAD (2) can also contain hydrophilic regions for titrations and/or reverse titrations (e.g. for detection and quantification of beta-lactam antibiotics), as well as user compliance regions for improving the accuracy of the quantitative analysis of the chemicals.

### Kits

The PADs (2) can be packaged in kits providing a user with all of the materials necessary for using the PAD (2). For example the kit can contain one or more PADs, vials, weight boats, and a centigram balance for preparation of samples. Instructions can be provided in hard copy, accessible via a link to a website or mobile application, accessible via a QR code or any combination thereof (including any equivalent and complementary instruction formats). The PADs (2) can be individually packaged and sealed in light, moisture, and/or oxygen resistant packets. Additionally the packets can be packaged with a desiccant in order to maintain a specific moisture level, and remove excess moisture.

### EXAMPLES

The following examples are provided to illustrate but not limit the invention disclosed herein by presenting preferred embodiments.

### Example 1

### Part Per Million Iodometric Titration on PAD

In a regular iodometric titration increasing amounts of thiosulfate is titrated into the sample until the equivalence point is reached according to equations (i)-(iv). In the saltPAD (2) iodometric titration as depicted in FIG. 1 each of the twelve reaction zones (8) is loaded with a different concentration of thiosulfate as set forth in Table 1 along with an excess of potassium iodide (16), acid (12) and starch indicator (10). Excess iodide reacts with iodate in the salt forming tri-iodide. If the amount of tri-iodide exceeds the amount of thiosulfate in the reaction zone on the PAD (2), the color of the starch indicator (10) will turn blue. In case that the amount of tri-iodide is smaller than the thiosulfate amount, the starch indicator (10) will be uncolored.

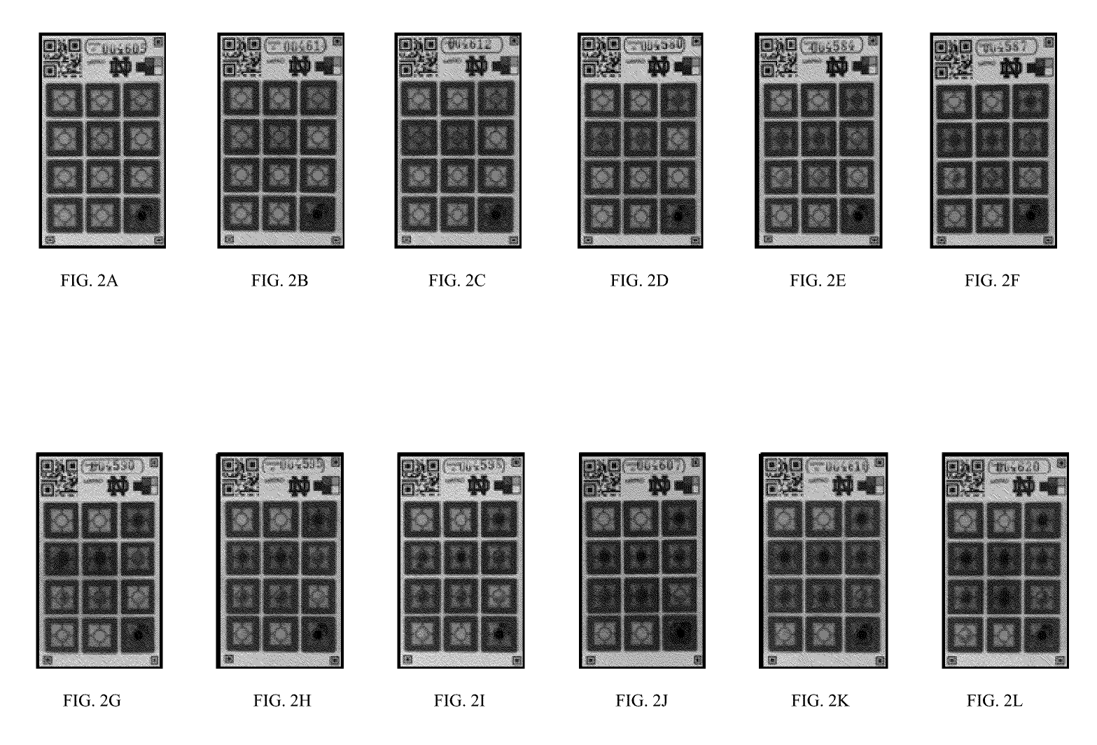

Eleven different levels of iodate amounts disposed on the saltPAD (2) were first interpreted by eye to determine the inter-operator precision, which is depicted in FIGS. 2 A)-2 L). The top left rectangular reaction zone (8) should always be white whereas the bottom right reaction zone (8) should always turn out dark blue. If this is not achieved, the test has to be re-run, which can be the quality of the saltPAD (2) that has been compromised. Thus simply comparing the number and the intensity with standard images that most closely match the saltPAD (2) response yields a rough estimate of quantification of the concentration of the iodate in the fortified salt which is demonstrated in FIGS. 2 A)-2 L), wherein each saltPAD (2) determined to contain from 0 to 11 ppm of iodate respectively. The test saltPAD (2) cards can be analyzed within 0.5 ppm on average and the inter-operator precision is determined to be 0.5 ppm. The inter-operator precision was determined by having two individual analysts analyze 110 saltPAD (2) test cards to determine this value.

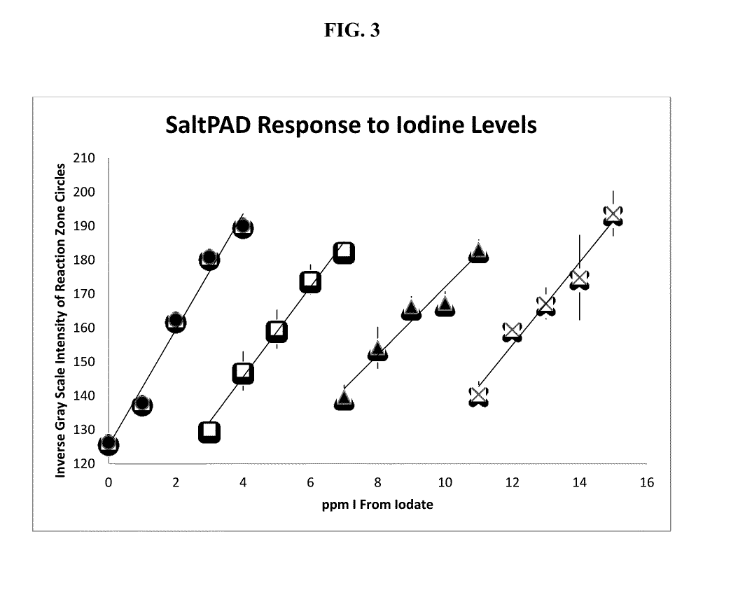

Now referring to FIG. 3, in order to determine the limit of detection (LOD) and the lower limit of quantification (LLOQ) of the present saltPAD (2) method, and to get a more precise determination of the iodate concentration, the color response of the saltPADs (2) were systematically tracked and analyzed by software image analysis. A photo of each saltPAD (2) test card was obtained using an iPhone 4 cell phone under controlled lighting conditions after a minimum of three minute reaction time to get a full color development on the saltPADs (2). Although the saltPAD (2) can be rotated in the cell phone's field, it must be in focus when the image is taken.

Secondly the fiducial markers (24) in the image are detected and located using the open-source ZXing package, and the fiducial markers (24) locations are used to transform the image to the canonical coordinate system to correct for perspective distortions. One is the affine transformation, which uses the positions of the three fiducial markers (24) in the QR code (4), while the other is the perspective transformation that uses the positions of the three fiducial markers (24) in the QR code (4) together with the position of the fiducial markers (26) in the three remainder corners on the saltPAD (2) opposite the corner, where the QR code (4) is located. Thirdly the image's white balance is corrected. Finally the color changes of the saltPAD (2) in the reaction zones (8) are identified to determine a quantitative test result.

Each reaction zone (8) shown in FIG. 3 displays a sigmoidal increase in color with increasing iodate concentration. While the flat regions of the curves (not shown) are not quantitatively useful and meaningful for determining iodate concentrations (but could be used for limit tests), the linear portions of the curves which cover a range of roughly 4 ppm of iodate are useful for such a purpose. Table 2 and FIG. 3 show that each curve represents a reaction zone that contains a different amount of thiosulfate. The curve depicted with solid circles contains 6 nmol of thiosulfate with a slope of 17.1±1.4 and y-intercept of 125.4±3.5, which represents the inverse gray scale intensity of reaction zone circles and a coefficient of determination R² of 0.980. The curve exemplified with open squares contains 33 nmol of thiosulfate with a slope of 13.3±0.8 and y-intercept of 92.5±4.3 and a coefficient of determination R² of 0.989. The curve demonstrated with solid triangles contains 60 nmol of thiosulfate with a slope of 9.9±1.3 and y-intercept of 72.1±11.9 and a coefficient of determination R² of 0.951. The curve graphed with solid squares contains 87 nmol of thiosulfate with a slope of 12.2±1.3 and y-intercept of 8.8±17.2 and a coefficient of determination R² of 0.966. As can be interpreted and concluded, the linear curves represent strong statistical models as the coefficient of determination R² values are close to 1, which is a perfect match for a statistical model. Each data point is an average of 36 reaction zones and FIG. 3 further depicts an error level of ± of one standard deviation.

Using line fitting parameters from the lower concentration range of the curve having 6 nmol of thiosulfate, the LOD was determined to be 0.6 ppm and the LLOQ was estimated to be 2.1 ppm. The saltPAD (2) test card contains three reaction zones in the 0-4 ppm range and three reaction zones in the 3-7 ppm range, plus reaction zones sensitive at 7-11 and 11-15 ppm ranges. The replicate measurements give superior precision at 1-7 ppm levels of iodizing agent. The 3 ppm value is particularly essential because after accounting for the 5-time dilution of the salt sample during preparation, this level differentiates salt that is properly iodized at least 15 ppm from salt that has been under-iodized.

TABLE 2

| Curve | S₂O₃²⁻ (nmol) | Slope | y-intercept | R² |
|---|---|---|---|---|
| Solid Circles | 6.0 | 17.1 ± 1.4 | 125.4 ± 3.5 | 0.980 |
| Open Squares | 33 | 13.3 ± 0.8 | 92.5 ± 4.3 | 0.989 |
| Solid Triangles | 60 | 9.9 ± 1.3 | 72.1 ± 11.9 | 0.951 |
| Solid Squares | 87 | 12.2 ± 1.3 | 8.8 ± 17.2 | 0.966 |

### Example 2

### Determination of the Accuracy and Precision of the PAD Method

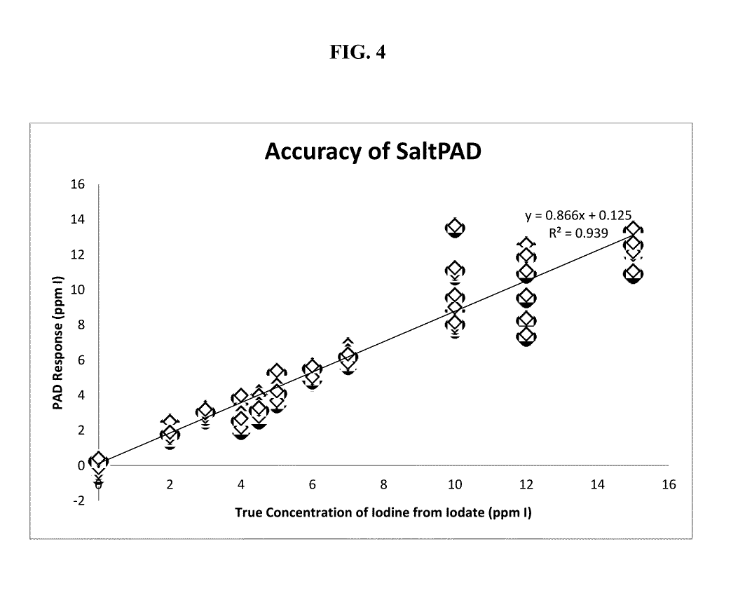

Now referring to FIG. 4 in order to determine important analytical parameters, the accuracy and precision of the saltPAD (2) and the method were analyzed. After construction of the calibration curves as illustrated in FIG. 3, sodium chloride brine was subsequently spiked with eleven different known amounts of iodate levels, and the samples were analyzed blind by two individual operators, who each applied test samples, ran the replicate saltPAD (2) test cards and subsequently evaluated the saltPAD (2) test card results independently for 5-fold dilution replicates. In total 110 saltPAD (2) test cards were used for the determination of the accuracy and the precision.

For determining the average absolute accuracy for the saltPAD (2) test cards, the following equation (1) was used:

$$\frac{{\sum }_{i = 1}^{n} \vert {X}_{real} - {X}_{measured} \vert}{n} . \qquad ( 1 )$$

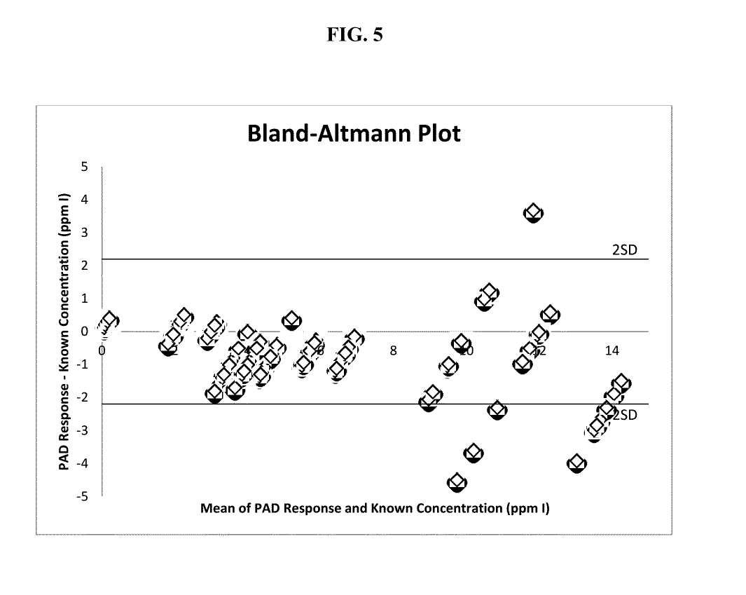

The Xᵣₑₐₗ is the known concentration of the different levels of the iodate, X_{measured} is the saltPAD (2) response as quantified by ImageJ software and n is the replicate number of the saltPADs (2) used. In the shown curve and working example in FIG. 4, n is 106, as four of the 110 saltPADs (2) resulted in discrepant results, wherein the color intensities did not fall within any of the calibration ranges, they were excluded from the data analysis. The average absolute accuracy for the saltPADs (2) for determination of iodine levels in the iodized salt were determined to be 0.9 ppm over the concentration range of 0.6-15 ppm. As can further be seen in FIG. 4, there is a high degree of positive correlation between the saltPAD (2) response and the true concentration of the iodine from the iodate, as the coefficient of determination R² has been calculated and determined to be 0.939. However in the determined values, there was found to be a 0.7 ppm systematic underestimation of the iodine concentration, as there was an inherent average bias of −0.7 ppm, which is further demonstrated in the Bland-Altmann plot as depicted in FIG. 5. One potential explanation for this systematic error lies in the camera software. Camera software often adjusts images to make them pleasing to the eye such as by adjusting the brightness. When a plurality of blue circles appear, the camera software brightens the image. A brighter image will lower the apparent iodine concentration. This underestimation bias is corrected through the use of a standard curve such as the one showed in FIG. 4. As can be seen in FIG. 5, only 9 of the analyzed samples were found to be 2 Standard Deviations (SD) away from the average saltPAD (2) response.

For determining the average precision for the saltPAD (2) test cards, the following equation (2) was used:

$$\frac{{\sum }_{i = 1}^{n} SD}{n} . \qquad ( 2 )$$

SD is the standard deviation of 10 replicates per iodate sample and n is the number of unknown samples. For iodate samples in the full range covering 0-15 ppm, the average precision was determined to be 0.9 ppm, whereas for samples in the range of 0-7 ppm, the average precision is 0.3 ppm.

For determining the average absolute inter-operator, the following equation (3) was used:

$$\frac{{\sum }_{i = 1}^{n} \vert {\overline{X}}_{1} - {\overline{X}}_{2} \vert}{n} . \qquad ( 3 )$$

wherein X̅₁ is the average response of 5 saltPADs (2) analyzed from analyst 1, and X̅₂ is the average response of 5 saltPADs (2) independently analyzed from analyst 2, and n is the total number of samples, which is 11. Visual comparison to standard images that most closely match the saltPAD (2) results in average absolute inter-operator precision of 0.5 ppm as determined and evaluated by equation (3).

### Example 3

### Determination of the Stability and Robustness of the PAD Method

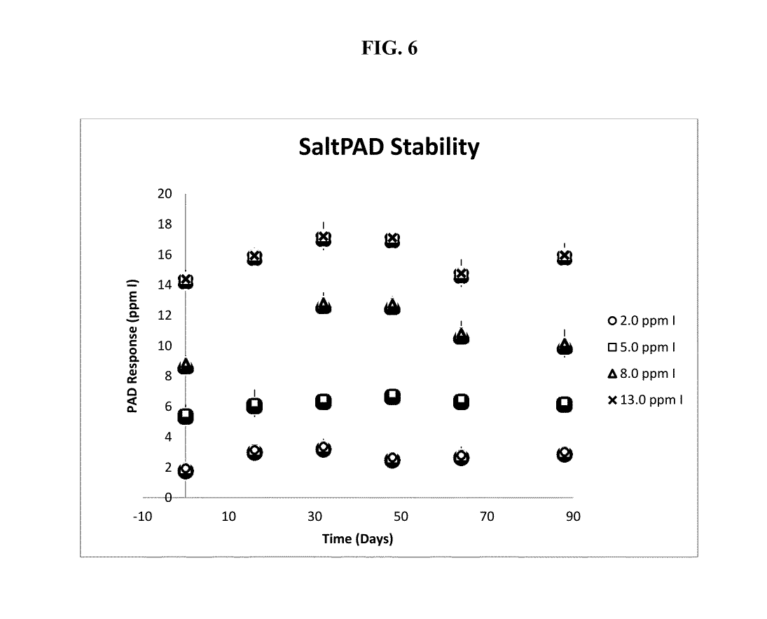

In order to test the stability and the robustness of the saltPAD (2), storage of the saltPADs (2) and different water sources were tested to evaluate the saltPAD (2) response, which is depicted in FIG. 6. SaltPADs (2) were packed in Ziploc bags and they were wrapped in aluminum foil to exclude light, and subsequently stored in a 40° C. convection oven for several weeks and up to a final time-point of 90 days. The saltPAD (2) response was tracked over time by analyzing a low, moderate, and high amount iodate standard level reflected by an iodate amount of 2.0, 5.0, 8.0 and 13.0 ppm respectively. An 8.0 ppm iodate standard was made up in a matrix of 3.7 M salt in tap water with a high mineral content, >180 ppm, and also in a matrix of water having a high content of natural organic matter. Each point is the average response of 3 saltPAD (2) test cards and also shown is a one standard deviation from the average saltPAD (2) response. The iodate standards that were made in hard water exhibited an error rate of 8% (n=2), whereas the standards with the high content of natural organic matter produced an error rate of 17% (n=2). But generally as can be viewed in FIG. 6, the saltPAD (2) response measured up to 90 days with 2.0, 5.0, 8.0 and 13.0 ppm iodate levels all show acceptably low standard deviations, and more importantly, the saltPAD (2) response which for all levels of 2.0, 5.0, 8.0 and 13.0 ppm iodate does not decrease significantly at the end of the 90-day testing period compared to the start-point at day 0. Thus the above data confirm the stability and robustness of the saltPAD (2) device and the method used to quantitate iodate levels in fortified salt compositions. As such the saltPAD (2) test card is not limited to only being used with extreme pure water but can similarly be employed with water that can not be ultra-pure. Ideally a quality check would be performed by using a water source as the solvent and diluent of a known stock standard.

### Example 4

### Utility of Iodometric Titration on PAD for Other Analyses

As explained previously iodometric titration on the PAD (2) is not just limited to the quantitation of iodate in fortified salt, but on the other hand, serves as a highly sensitive chemical analytical procedure for iodometric titration of beta-lactam antibiotics as well. In this procedure, the beta-lactam is first hydrolyzed in base where after the solution is acidified, and excess iodine is added to react with the sulfur group in the degraded antibiotic, and then subsequently back-titrated with sodium thiosulfate. This is illustrated below in reaction (v):

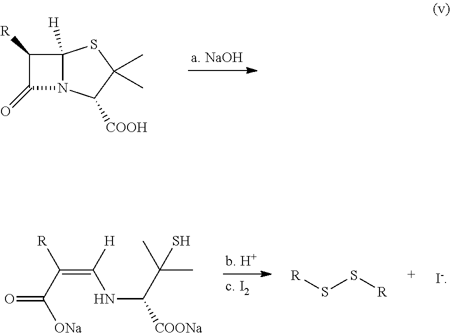

The formed tri-iodide subsequently reacts with the starch indicator to form the blue color on the PAD (2) according to reaction (iv) previously illustrated.

A finished pharmaceutical pill sample comprising ampicillin and a secondary standard of ampicillin were reacted and taken through the chemical transformations as depicted in FIGS. 2-3 in glassware, and as described in the sections, “Reagents and Reagent Deposition” and “Deposition of the Composition to be Analyzed on the PAD”. The resulting solutions were diluted 1:50 in water in order to get the iodine concentration within the working range of the PAD (2) analytical method. By comparing the blue color intensity development of the standard solution to the pill solution, it was found that the pill contained 556 mg ampicillin as determined by the PAD (2) method compared to the glassware titration method, wherein the amount of the ampicillin in the pill was found to be 497 mg. Thus determination of the ampicillin amount by the PAD (2) method only resulted in a 12% deviation from the glassware titration method.

### Example 5

### Quality Control of SaltPad Production

A quality control check was performed on each batch of saltPAD that was spotted by the Biomek FX 96-well-plate replicating robot. The quality control check included selecting 3 saltPADs from the beginning of the batch, 3 from the middle and finally 3 saltPADs from the end of the batch and running a 2.0, 5.0, and 8.0 ppm iodate standard solution on one saltPAD from each section. After at least three minutes, visual inspection and evaluation by comparison to standard images indicated whether the saltPADs had been consistently spotted throughout the batch. No irregularities at these tested amounts of iodate were discovered on the saltPADs.

For the sake of brevity it should be understood that certain structures and functionality, or aspects thereof, of embodiments of the presently disclosed PAD that are evident from the illustrations of the Figures have not been necessarily restated herein. Also additional features relating to the use of the PAD can be found in U.S. patent application Ser. No. 13/566,915, filed Aug. 3, 2012, the entire disclosure of which is expressly incorporated herein by reference herein.

In sum, it is to be understood and realized that since numerous modifications and changes will readily be apparent to those having ordinary skill in the art it is not desired to limit the claimed invention to the exact entities as specifically demonstrated in this disclosure. Accordingly all suitable modifications and equivalents can be resorted to falling within the scope of the claimed invention. Thus it should be understood that various features and aspects of the disclosed of the disclosed embodiments can be combined with or substituted for one another in order to form varying modes of the claimed invention.

Unless defined otherwise, all technical and scientific terms used herein have same meaning as commonly understood by the person of ordinary skill in the art. As used herein and in the appended claims, the singular form “a”, “and”, and “the” include plural referents unless the context clearly dictates otherwise. All technical and scientific terms used herein have the same meaning. Thus the scope of the embodiments of the claimed invention should be determined by the appended claims and their legal equivalents rather than by the Figures.

What is claimed is:

1. A paper analytical device for detection of part per million levels of iodide and quantification of part per million levels of iodate by iodometric titration comprising:
a porous hydrophilic medium;
a plurality of reaction zones associated with the porous hydrophilic medium;
at least one assay reagent which has been deposited and dried onto each of the at least reaction zones;
at least one electronically readable information zone which provides a color information for the detection and quantitation of part per million levels of iodate in fortified salt when a predetermined solution of the salt has been applied onto the reaction zones,
wherein the at least one electronically readable information zone comprises a plurality of fiducial markers for correct alignment of the paper analytical device for transforming and correcting a captured image such that analysis and processing of the color information to more accurately quantitate part per million levels of iodate is efficiently afforded.

2. The paper analytical device of claim 1, wherein the plurality of the fiducial markers have been constructed and are in communication with at least one of an identification tag of a two-dimensional barcode, and wherein the plurality of the fiducial markers are further located on the paper analytical device as individual units separated from the two-dimensional barcode.

3. The paper analytical device of claim 1, further comprising at least one color calibration zone for detection and quantification of part per million levels of iodate.

4. The paper analytical device of claim 1, wherein at least 5 to 16 reaction zones are present on the surface of the device.

5. The paper analytical device of claim 1, wherein each of the reaction zones are further divided into a plurality of separate regions, wherein each region has a different dried assay reagent applied thereon.

6. The paper analytical device of claim 5, wherein between 3 and 9 regions are present in each reaction zone.

7. The paper analytical device of claim 5, wherein a region in each reaction zone includes a color indicator.

8. The paper analytical device of claim 7, wherein the color indicator is starch and is used to make the detection of iodate in fortified salt more sensitive by forming a blue complex when conducting the iodometric titration.

9. The paper analytical device of claim 7, wherein additional regions in each of the reaction zones have varying concentrations of sodium thiosulfate, a fixed concentration of p-toluenesulfonic acid and a fixed concentration of a mixture of potassium iodide with cadmium chloride applied thereon.

10. The paper analytical device of claim 9, wherein the varying concentrations of sodium thiosulfate cover a range from at least approximately 3.0 mM to 150 mM.

11. The paper analytical device of claim 1, further including one negative and at least one positive control.

12. The paper analytical device of claim 11, wherein the positive control is potassium iodate.

13. The paper analytical device of claim 6, further comprising a hydrophobic barrier that is present to define each of the reaction zones such that when a solution to be tested is applied to the reaction zones, the reagents present on the separate regions subsequently dissolve and mix with the solution.

14. The paper analytical device of claim 1, wherein the chemical components to be quantified further include degradation products of beta-lactams.

15. A method for detection and quantification of part per million levels of iodate by iodometric titration comprising:
i) providing a paper analytical device according to claim 1;
ii) making a dilution of the fortified salt in water,
iii) disposing the solution drop-wise onto the at least twelve reaction zones;
iv) shaking the paper analytical device gently for at least a predetermined amount of time such that the reaction zones turn blue if a preset iodate concentration is exceeded; and
v) analyzing the color information by image analysis or by eye so as to quantitate the amount of iodate.

16. The method of claim 15, wherein the method further comprises automating the color analyzing by:
a) capturing an image of the paper analytical device by a camera device; and
b) providing an image analysis software in which the image analysis software is capable of quantitating a color change within the at least twelve reaction zones of the paper analytical device that is depicted in the captured image.

17. The method of claim 16, wherein the paper analytical device further comprises a plurality of fiducial markers for correct orientation and alignment of the captured image of the paper analytical device, wherein the image analysis software transforms and corrects the captured image according to the plurality of fiducial markers such that analysis and processing of the color information so as to quantitate part per million levels of iodate is afforded by aligning the captured image with compiled images in a database, reading a test response from the reaction zones in transformed image, and classifying the test results.

18. The method of claim 17, further comprising providing the image analysis on the camera device for processing the image in situ, or on a network server, wherein the captured image is processed by sending the image to the network server and the network server is configured to execute the analysis and subsequently send the detection and quantification results back to the camera device.

19. The method of claim 15, wherein the paper analytical device quantifies amounts of 0.6-15 ppm iodine in salt solutions.

20. The method of claim 16, wherein the image analysis quantifies iodine with an average absolute accuracy and precision of 0.9 ppm over an iodine level range of 0.6-15 ppm in the salt solution.

21. The method of claim 15, wherein a visual comparison to standard images results in an average absolute accuracy and precision of 0.5 ppm.

22. The method of claim 15, wherein the method has a limit of detection of at least 0.6 ppm and a lower limit of quantification of at least 2.1 ppm.
